(19) United States
(12) Patent Application Publication
(10) Pub. No.: US 2005/0159693 A1
(43) Pub. Date: Jul. 21, 2005

(54) Arm/shoulder brace for shoulder subluxation

(76) Inventor: Wayne Sims, Tiskilwa, IL (US)

Correspondence Address: Wayne Sims;R.R. #1, 18213 1250 N Ave., Tiskilwa, IL, 61368, US

(21) Appl. No.: 10/759,862

(22) Filed: Jan. 20, 2004

(51) Int. Cl. A61F 5/00

(52) U.S. Cl. 602/20

(57) ABSTRACT

The invention is made of two fully adjustable aluminum pieces (or other similar product) whereby one piece is vertical and the other piece is horizontal. The pieces are connected together near the patient's waistline and connected to the patient with a belt clip. The two pieces form a brace. The vertical piece is topped with foam padding which fits in the armpit of the patient's injured shoulder to provide support to the injured shoulder. The horizontal piece allows for either an injured arm or an uninjured arm to rest horizontally and forms an armrest for the injured patient. The horizontal position of the arm keeps the arm and shoulder in a comfortable position for the patient during recovery from injury or surgery.

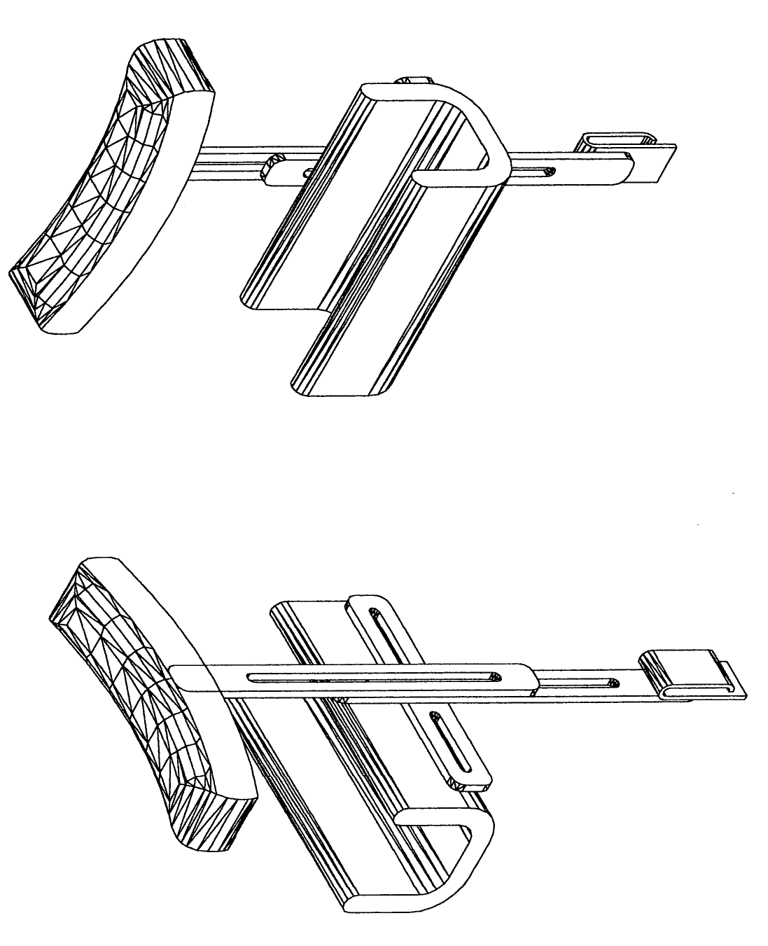

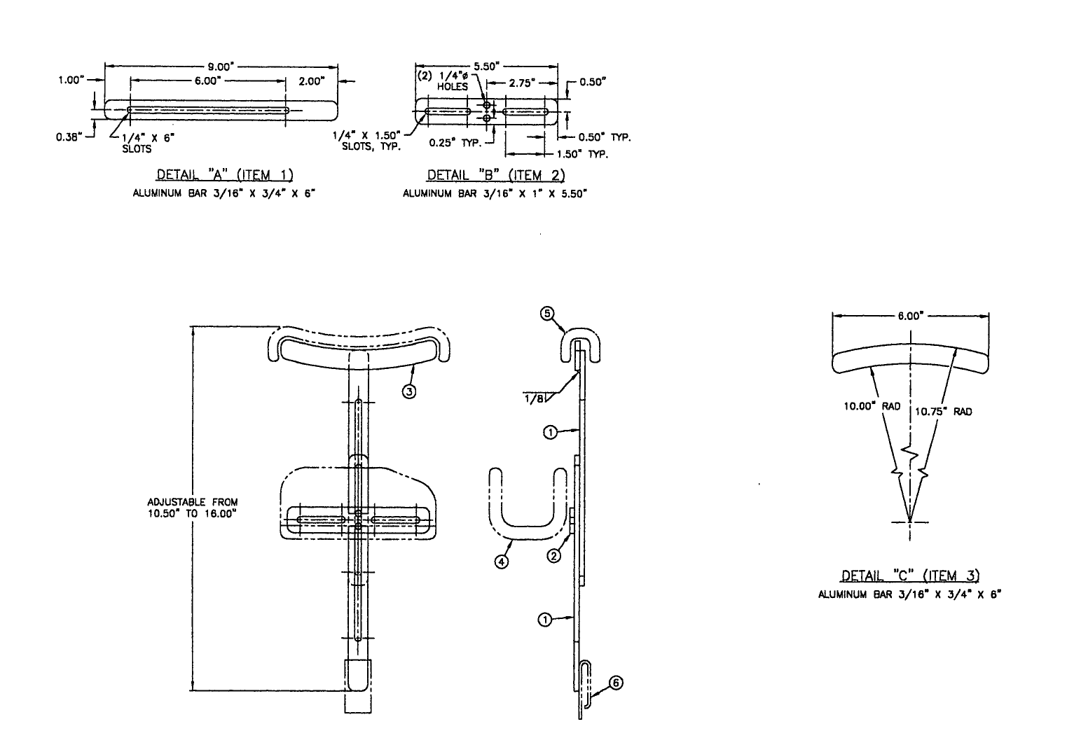

### BACKGROUND OF THE INVENTION

This invention relates generally to medical devices. More particularly, the present invention concerns an improved shoulder and arm brace to stabilize a shoulder during recovery from an injury or surgery to limit pain and stress to the injured shoulder.

### DISCUSSION OF PRIOR ART

Previous shoulder braces did not secure the shoulder in place while providing an armrest for the injured arm. This brace can effectively keep the shoulder at a prescribed position as determined by the patient's physician while providing stabilization for the shoulder and arm at a comfortable position.

### BRIEF SUMMARY OF THE INVENTION

The invention is to provide a stabilizing brace for the shoulder and an armrest, during recovery from an injury or surgery, to reduce pain to the patient. The invention will provide shoulder subluxation, support, and assist in recovery process as well.

### DETAILED DESCRIPTION

The invention consists of three pieces of aluminum or similar product that are fully adjustable. These pieces make the shape of a cross at the base of the armrest. The armrest and vertical bar can be adjusted for various numeral lengths. The armrest can be a half-cast secured to the brace, or in case of a fractured arm, a channel of aluminum can be secured to the brace, in which to rest the cast. The vertical brace is topped with a crutch type foam pad which sits under the patient's armpit to hold the injured shoulder in place to reduce pain. The foam pad is placed under the patient's axilia. The brace is secured to the patient's waist with a belt clip.

1. What I claim as my invention is an arm/shoulder brace to stabilize and support an injured shoulder subluxation. The invention consists of a cross of two fully adjustable lengths of aluminum pieces (or similar product) whereby the vertical piece is topped with a foam pad, similar to a crutch, which fits under the patient's axilia. The horizontal piece is used to rest the arm and can also accommodate a cast in the event that the arm is also injured. The vertical and horizontal pieces are secured to the patient with a belt clip. The invention is primarily designed to reduce patient pain following an injury or surgery.
